(19) United States
(12) Patent Application Publication
(10) Pub. No.: US 2016/0158509 A1
(43) Pub. Date: Jun. 9, 2016

(54) ACTIVELY TRACKED MEDICAL DEVICES

(71) Applicant: IMRICOR MEDICAL SYSTEMS, INC., Burnsville, MN (US)

(72) Inventors: Steven R. Wedan, Savage, MN (US); Thomas W. Lloyd, Eagan, MN (US); Bryan A. Brutlag, Minneapolis, MN (US); Gregg S. Stenzel, Victoria, MN (US); Scott Kimmel, Roseville, MN (US)

(21) Appl. No.: 14/907,724

(22) PCT Filed: Jul. 29, 2014
(86) PCT No.: PCT/US2014/048583

§ 371 Date: Jan. 26, 2016

Related U.S. Application Data
(60) Provisional application No. 61/859,528, filed on Jul. 29, 2013.
(60) Provisional application No. 61/974,700, filed on Apr. 3, 2014.

(51) Int. Cl. A61M 29/00; G01R 33/28; A61B 17/34

(52) U.S. Cl. CPC A61M 29/00; A61B 17/34; G01R 33/287; G01R 33/286

(57) ABSTRACT

An actively tracked medical device comprising: a dilator having an inner tubular main body having a distal end and a proximal end, said tubular main body including at least first and second receiving channels positioned in a spaced apart relationship on an outer surface of said tubular main body; a region at the distal end of said tubular main body for supporting one or more tracking coils; an atrumatic tip portion operably coupled and positioned distal to said main body; a lumen extending through said tubular main body, said tip support and said atraumatic tip portion; and an outer polymer body having first and second ends, said outer polymer body operably covering said inner tubular main body and said tracking coils, said first end terminating adjacent a proximal end of said atraumatic tip portion and said second end terminating adjacent said hub.

### FIELD OF THE INVENTION

This invention relates to actively-tracked medical devices. More particularly, the present invention is related to a steerable sheath used in interventional vascular procedures to deliver tools into the human body, a dilator used in conjunction with the steerable sheath and a transseptal puncture device, which can be actively visualized and/or tracked in a magnetic resonance imaging (MRI) environment.

### BACKGROUND OF THE INVENTION

MRI has achieved prominence as a diagnostic imaging modality, and increasingly as an interventional imaging modality. The primary benefits of MRI over other imaging modalities, such as X-ray, include superior soft tissue imaging and avoiding patient exposure to ionizing radiation produced by X-rays. MRI's superior soft tissue imaging capabilities have offered great clinical benefit with respect to diagnostic imaging. Similarly, interventional procedures, which have traditionally used X-ray imaging for guidance, stand to benefit greatly from MRI's soft tissue imaging capabilities. In addition, the significant patient exposure to ionizing radiation associated with traditional X-ray guided interventional procedures is eliminated with MRI guidance. Presently, however, due to the lack of appropriate surgical instrumentation MRI is not available to interventionalists for use during interventional therapy to accurately track and precisely guide medical devices to regions of a patient needing treatment.

By way of example, the left atrium of the heart is the most difficult cardiac chamber to access percutaneously. Although the left atrium may be reached via the left ventricle and mitral valve, manipulation of catheters requiring two 180 degree turns may be cumbersome and time consuming for the surgeon. Thus the transseptal puncture is the procedure of choice because it permits a direct route to the left atrium via the intra-atrial septum and systemic venous system. The technique has been used for mitral valvuloplasty and ablation in the left heart and with the explosion of interest in catheter ablation of atrial fibrillation, transseptal puncture is increasingly being adopted by cardiac electrophysiologists and the method of choice. However, it is critical for cardiac electrophysiologists interested in transseptal puncture techniques to be able to track sheaths for carrying dilators and transseptal puncture devices in an MR environment to know when the transseptal device has punctured the septum to avoid unintentional perforation of the opposite side of the heart.

While there are many types of sheaths, dilators and transseptal needles currently available for transseptal puncture (and other medical procedures) few are well-suited for use in an MRI environment and to the inventor's knowledge none are actively tracked. For example, deflectable (i.e., steerable) sheaths including multi-directional, bi-directional and uni-directional deflectable catheters are known. However, many of these sheaths have ferromagnetic components that pose a safety hazard to the patient in a magnetic field environment, as they can cause injury to the patient, as they may move in an undesired manner due to the magnetic field. The ferromagnetic components can also cause image distortions, thereby compromising the effectiveness of the procedure. Still further, such sheaths may include metallic components that may cause radiofrequency (RF) deposition in adjacent tissue and, in turn, tissue damage due to an extensive increase in temperature.

Similarly, dilators and transseptal needles currently available have the same problems, i.e. they either include ferromagnetic components that cause image distortions or include metallic components that cause RF deposition in tissue.

Moreover, it is difficult to track and/or visualize the location of the aforementioned sheaths, dilators and transseptal needles in an MRI environment. In general, there are two types of tracking in an MRI environment: active tracking and passive tracking. Active tracking is more robust than passive tracking but typically involves resonant RF coils that are attached to the device and directly connected to an MR receiver allowing for the determination of the three-dimensional coordinates of the resonant RF coils within the scanner. To the inventors' knowledge neither active nor passive tracking techniques are presently utilized in conventional sheaths, dilators or transseptal needles.

Thus, there is a need for sheaths, dilators and transseptal needles that can be used alone or in combination with each other that can be actively and effectively tracked and/or visualized in an MRI environment.

### BRIEF SUMMARY OF THE INVENTION

The shortcomings of the present steerable sheaths, dilators and transseptal needles are addressed by the actively tracked medical devices in accordance with the invention. The actively-tracked medical devices in accordance with the invention are directed to a deflectable tip sheath, a dilator and a transseptal needle that may be used alone or in combination with each other.

The actively-tracked medical device in accordance with the invention substantially obviates the deficiencies and disadvantages associated with the related art as set forth above. More specifically, the present invention is directed to a deflectable sheath, a dilator and a transseptal needle that can be actively tracked in an MRI environment, without excessive RF deposition (i.e., local tissue heating) and the other safety and procedural drawbacks associated with the prior related art.

In one aspect of the invention, a deflectable sheath that can be easily and effectively visualized and actively tracked in an MRI environment is provided.

In another aspect of the invention an actively tracked dilator that is used in conjunction with a deflectable sheath and effectively visualized and tracked in an MRI environment is provided.

In yet another aspect of the invention a deflectable sheath and/or dilator that is effective when used in an MRI environment and does not, among other things, distort the image, and does not cause local tissue damage due to excessive RF deposition along the length of the catheter is provided.

In yet another aspect of the invention, a transseptal puncture device that may be used in combination with the aforementioned actively-tracked, deflectable sheath and/or dilator is also provided.

### BRIEF DESCRIPTION OF THE DRAWINGS

For a better understanding of the invention, and to show how the same may be carried into effect, reference will now be made, by way of example, to the accompanying drawings, in which:

FIG. 1 is a perspective view of a steerable sheath in accordance with one aspect of the invention.

FIG. 2 is a perspective view of a dilator with integrated tracking components in accordance with one aspect of the invention.

FIG. 3 is a perspective view of one aspect of the dilator shaft in accordance with the invention.

FIG. 4 is a perspective view of the distal end of the dilator in accordance with the invention.

FIG. 5 is a perspective view of the distal end of the dilator in accordance with the invention.

FIG. 6 is a perspective view of one aspect of the dilator hub in accordance with the invention.

FIG. 7 is a perspective view of another aspect of the dilator hub in accordance with the invention.

FIG. 8A is a perspective view of a transseptal needle in accordance with one aspect of the invention.

FIG. 8 B is a cross-section view of the shaft of the transseptal needle of FIG. 8A taken along line AB-AB.

FIG. 8C is an enlarged detailed view of area A of the transseptal needle of FIG. 8A.

FIG. 9 is a perspective view of the transseptal needle in accordance with the invention depicting one aspect of the transseptal needle.

FIG. 10 is a perspective view of the transseptal needle in accordance with the invention depicting another aspect of the transseptal needle.

FIG. 11 is a perspective view of the transseptal needle in accordance with the invention depicting another aspect of the transseptal needle.

FIG. 12 is a perspective view of the transseptal needle in accordance with the invention depicting one aspect of the distal tip.

FIG. 13 is a perspective view of the transseptal needle in accordance with the invention depicting another aspect of the distal tip.

FIG. 14-17 are perspective views of the transseptal needle in accordance with the invention depicting various aspects of the distal tip.

FIG. 18A is a view of a transseptal needle in accordance with the invention placed inside a deflectable dilator.

FIG. 18B is an enlarged detailed view taken of area A of FIG. 18A showing the transseptal needle placed proximal to a bend in the dilator.

### DETAILED DESCRIPTION OF THE INVENTION

Numerous structural variations of an MR compatible steerable sheath and dilator that can be actively tracked in accordance with the invention are contemplated and within the intended scope of the invention. Those of skill in the art will appreciate that the exemplary actively tracked sheath and/or dilator can be accomplished in a variety of ways. Those of skill in the art will also appreciate that the transseptal puncture device may comprise numerous structural variations. Therefore, for purposes of discussion and not limitation, exemplary embodiments of the actively tracked steerable sheath, dilator and transseptal puncture device will be described in detail below.

Actively tracking the medical devices in accordance with the invention may be accomplished by integrating one or more tracking coils into the sheath or the dilator. Tracking coils may include wound tracking coils or printed circuit board (PCB) tracking coils. Active tracking may also be accomplished by integrating tracking coils into the dilator and using the actively tracked dilator to track both the sheath and transseptal needle in in vivo MRI applications. In alternative embodiments, tracking coils may also be incorporated into the transseptal needle.

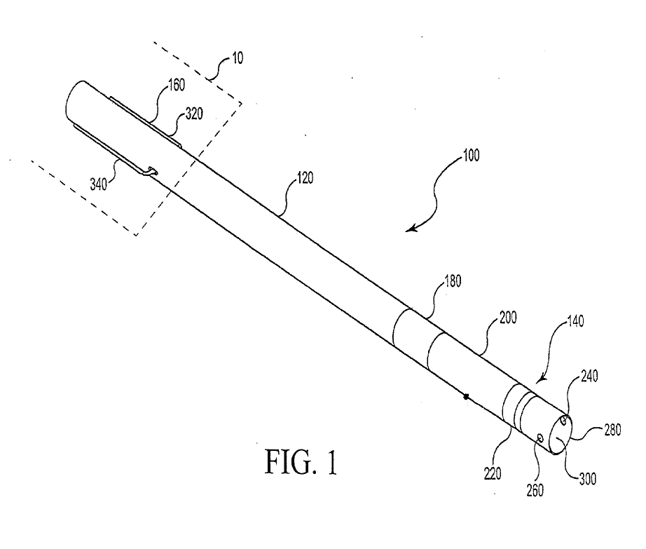

Referring now to FIG. 1 the steerable sheath 100 that may be used in combination with the dilator and transseptal needle in accordance with the invention will now be explained. Steerable sheath 100 may be used in an MRI environment to deliver a variety of tools such as catheters, guide wires, implantable devices, etc. into cavities and passageways of a patient body. The steerable sheath 100 includes a deflectable tip portion 200 that is able to bend at an about 180 degrees offset from the longitudinal axis of the catheter sheath 100. This flexibility allows the medical professional to makes very tight turns to deliver the aforementioned tools to the cavities and passageways of the patient body.

Referring again to FIG. 1 a perspective view of an MR compatible steerable sheath that is suitable for use in an MRI environment is depicted. The MR compatible steerable sheath 100 in accordance with the invention broadly includes tubular shaft 120 with distal 140 and proximal ends 160. Tubular shaft 120 includes an outer diameter, an inner diameter and defines a central lumen 300 therewithin configured to receive, for example, a dilator. Tubular shaft may be constructed of a variety of polymers including polyether block amides such as PEBAX (Arkema), polyurethane, nylon, derivatives thereof and combinations of the foregoing.

Tubular shaft may include two or more lumens. One lumen may comprise the main lumen that allows for the passage of the transseptal needle as well as fluid such as contrast and saline. Additional lumens may be used to house transmission lines that connect the tracking region to the proximal hub. Tubular main shaft may also be constructed of two tubes, an inner tube that has a main lumen as well as two 180 degree opposed channels that receive the transmission lines and an outer tube that slides over the inner tube and transmission lines.

In an alternative embodiment an inner tube that has a simple circular profile may create the main lumen for the dilator and includes an outer tube that has a profile such that it slides over the inner tube, but also contains 180 degree channels that receive the transmission lines.

The main tubular shaft may also comprise a single tube that is reflowed over the transmission lines. The proximal hub could be connected to the main shaft by an over molding process or with adhesive. The main shaft could be connected to the tracking region by a reflow process or with adhesive.

Distal end 140 includes transition section 180, deflectable tip portion 200, and metal ring 220. Those of skill in the art will appreciate that metal ring 220 may comprise a metal foil. Metal ring 220 may be provided at the deflectable tip portion for spoiling the active tracking signal on a medical device (such as a dilator) inserted into sheath 100 and for identifying and/or tracking the tip 280 of the sheath. Spoiling the tracking signal from a device inserted in the sheath at a specific and limited location along the sheath provides a method for identifying that location on the sheath during active MR tracking.

Pressure relief holes 240, 260 may be formed in the tubular shaft 120 at the distal end 140. Those of skill in the art will appreciate that while only two pressure relief holes 240, 260 are shown there may any number of pressure relief holes formed and still be within the scope of the invention. When retracting an item housed by the sheath 100, such as a dilator, catheter or MR active tracking system, pressure may form at the end of the sheath thereby drawing or sucking in tissue. Pressure relief holes 240, 260 are designed to reduce this pressure thereby ameliorating the risk of tissue damage.

Transition section 180 is optionally included for purposes of manufacturability. The deflectable tip section 200 has a significantly lower durometer making it more malleable and flexible than the main body portion of tubular shaft 120 which has a higher durometer or, in other words, quite stiff. As a consequence, these two sections do not bond to one another well. Transition section 180 has a mid-range durometer allowing it to bond well to both the deflectable tip section 200 and the main body of the tubular shaft 120. Those of skill in the art will appreciate that the transition section 180 may be of any length desired so as to provide an adequate transition between the distal tip portion 200 and the main body portion 170. In one exemplary embodiment transition section may range from about 0.25 to about 0.75 inches. In addition, those of skill in the art will appreciate that transition section may be eliminated and the deflectable tip section 200 may be coupled to the main body of tubular shaft 120 by means known to those of skill in the art without departing from the spirit of the invention.

Steerable sheath 100 includes central lumen 300 therewithin. In one aspect of the invention, the inner diameter of the tubular shaft 120 is approximately 6 French or greater but those of skill in the art will appreciate that varying internal diameters may be used depending on the particular application and instrumentation required without departing from the scope of the present invention. Central lumen 300 may include one or more liners (not shown) disposed therewithin to allow for easier movement of instruments therethrough. Liners may comprise materials made from polytetrafluoroethylene (PTFE), fluorinated ethylene propylene copolymer (FEP), nylons and combinations of the foregoing. Alternatively, the lumen 300 may be coated with any such polymers. The polymer tubular shaft 120 may also include one or more passive visualization markers, such as a ferrous or magnetic marker (not shown), disposed circumferentially about the tubular shaft 120 at one or more locations along the length thereof and/or one or more active visualization markers such as an active tracking coil along the length of the tube. An active tracking coil may comprise one or more small antennas integrated into the device and include traces on a circuit board, coiled wire, and/or a dipole. If an active visualization marker is used, one or more devices may be included in the conductors to mitigate RF field heating may be included. Such devices include chokes, transformers, impedances, and other such devices known to those of skill in the art. One or more fluoroscopy markers (not shown) may also be included along the length of the polymer tubular shaft 120. Alternatively, an active tracking device may be eliminated from the sheath and instead be integrated into the dilator. The dilator may then be used to track the location of the sheath as described below.

One or more optional fluid ports (not shown) may be located on the proximal end 160 of the tubular shaft 120 to allow for homeostasis of the sheath with the patient body. The fluid port(s) allows access for the user or physician to aspirate blood from the steerable sheath lumen 300 and flush with saline. Aspirating and flushing of the sheath prevents air from entering the body before and during insertion of a dilator, tool and/or other instrumentation.

In addition, or as an alternative to active/passive tracking of the sheath, measurements taken from a dilator (or catheter) located within the sheath may be used to determine the location of the tip of the sheath. The change in the electrogram signal (in the case of a catheter) or tracking signal (for any actively tracked device) may then be used to manually or automatically mark the sheath tip on the MR image and/or an associated visualization/navigation tool. In other words, when a tracking coil on an actively tracked device exits the sheath the amplitude of the tracking signal will increase. In the case of a catheter with electrodes, the measured electrogram amplitude between of the one or more electrodes increases while impedance decreases and differs from when the catheter is inside the sheath. Variation in either electrogram or tracking signal measurements can be used for tracking of the sheath tip.

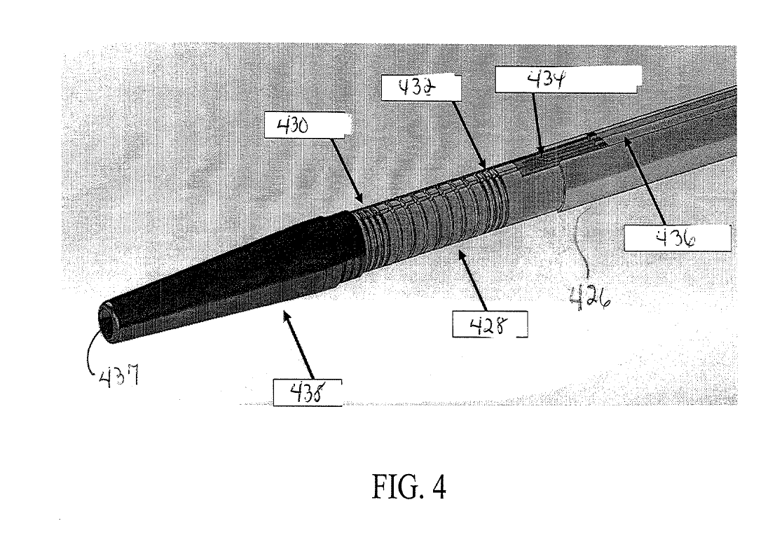

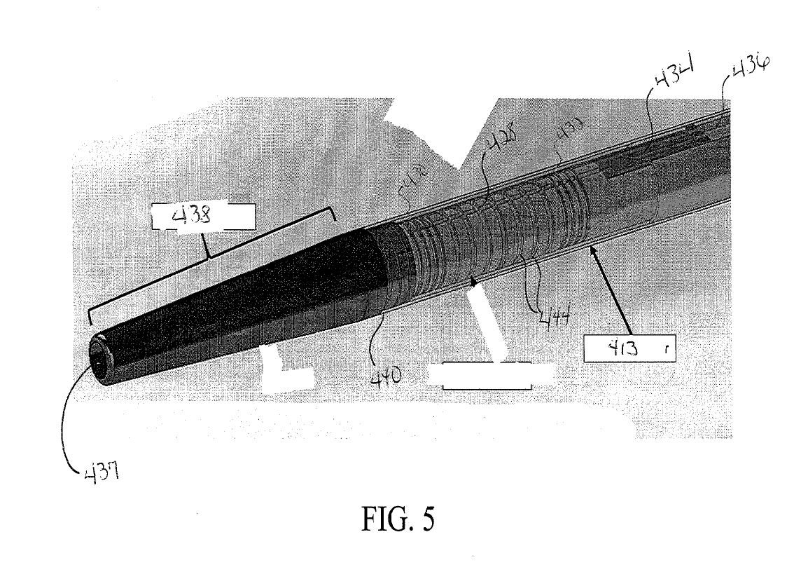

In an alternative embodiment, one or more tracking coils may be integrated into a dilator (as best seen in FIGS. 4 and 5) that is received within the sheath 100 and a conductive structure may be concentrically place inside the sheath. For example, such a conductive structure may comprise a metallic or gold foil. As the dilator is advanced through the sheath the metallic or gold foil will act to detune the tracking coil on the dilator and block it from receiving the tracking signal. Thus the tip of the sheath may be marked on an MR image or in an associated mapping tool.

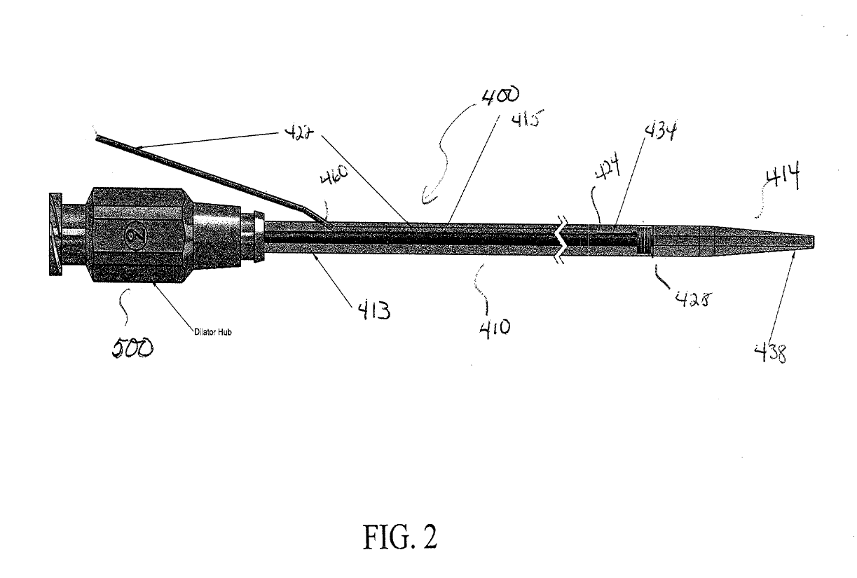

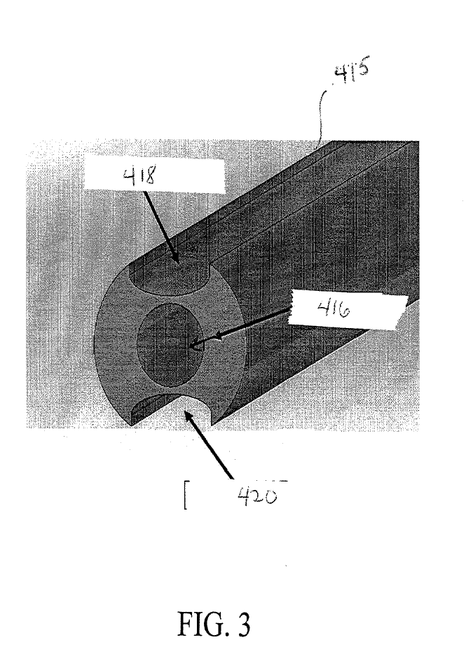

Referring now to FIGS. 2 through 7 the tracking dilator 400 in accordance with the invention will now be described. Tracking dilator 400 broadly includes tubular main body 410, proximal dilator hub 500 and distal tip 414. As best seen in FIGS. 2 and 3, tubular main body 410 includes an inner polymer body/multi-lumen extrusion construct 415 that is encapsulated by an outer, over-molded polymer body 413 as hereinafter described. The multi-lumen extrusion construct 415 includes primary lumen 416 therewithin and first and second channel lumens 418, 420. As can be seen, first and second channel lumens 418, 420 may be positioned on main body 410 in a spaced-apart relationship. As shown in FIG. 3 the spaced-apart relationship is 180 degrees from each other but may be any spaced-apart configuration. Primary lumen 416 is adapted to receive a transseptal needle, stylet, guide wire, fluid and/or contrast media. First and second channel lumens 418, 420 are adapted to receive tracking components such as a co-axial cable, transmission lines, matching networks and transmission line transformers. Those of skill in the art will appreciate that the C-shape in cross section channel configuration facilitate the placement of the transmission lines and other cables along the main body 410 of the dilator 400. Multi-lumen extrusion construct 415 may be molded from appropriate polymers including polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and the like.

Referring again to FIG. 2, outer polymer body 413 may be concentrically over-molded onto to the inner polymer body 415 to encapsulate the electronics as well as the inner polymer body 410. The resulting inner polymer body 415 may act as both a support for the tracking components, as hereinafter described, and include primary lumen 416 through which a stylet, guidewire, transseptal needle, fluid, and/or contrast and the like may be passed. The tracking components may include a coaxial cable 422 that may exit the dilator 400 adjacent the dilator hub 412 and includes an appropriate termination point 424 on the first or second channel lumens 418, 420. The inner polymer body 415 may comprise the same polymer that is used for the outer polymer body 413 and maybe reflowed to form one solid integral piece. Alternatively, the inner polymer body 415 may be slidably removable from the outer polymer body 413. The tracking region may also comprise a flat flex circuit that integrates the tracking coils and matching network cards

As best seen in FIG. 4, the distal end 426 of the multi-lumen extrusion or inner polymer body 410 includes optional tip support portion 428. Optional tip support 428 operably receives one or more tracking coils 430, 432 and matching network card 434. Alternatively, the network card or cards may be positioned within one or both channel lumens 418, 420. Alternatively, the network card or card may be partially supported by the tip support 428 and partially supported by one (or both) of the channel lumens 418, 420. Transmission line 436 may be operably received within first or second channel lumens 418, 420. Tip support 428 is bonded at its distal end to dilator tip 438 which is molded out of an appropriate polymer selected from polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and combinations of the foregoing, such that the resulting tip 438 is atraumatic. In another aspect of the invention, the dilator tip 438 may be bonded to or over-molded onto the tip support 428.

Referring to FIG. 5 after the tip support 428 has been bonded or over-molded onto the dilator tip 438 and one or more transmission lines 436 and other tracking components have been positioned in first and second channel lumens 418, 420 of the main body 415 of the dilator, an outer polymer coating, which forms the outer polymer body 413, may be over-molded onto the inner polymer body 415 as hereinbefore described. The outer polymer coating or body 413, encapsulates the transmission lines 422, 436 (coaxial cable and transformers), tip support area 428, matching network card 434 and one or more tracking coils 430, 432. The outer polymer body 413 extends from the distal end of the dilator hub 500 to the proximal end 440 of the dilator tip 438, to provide a continuous and smooth outer surface the entire length of the dilator shaft. The outer polymer coating/body 413 may be bonded or reflowed to the proximal end 440 of the dilator tip 438. Tip support 428 includes a plurality of grooves 444 circumferentially positioned on tip support 428. Grooves 444 may encourage or improve the mechanical bond between the tip support and the outer polymer coating/body 413. Grooves 444 may also support one or more tracking coils 430, 432.

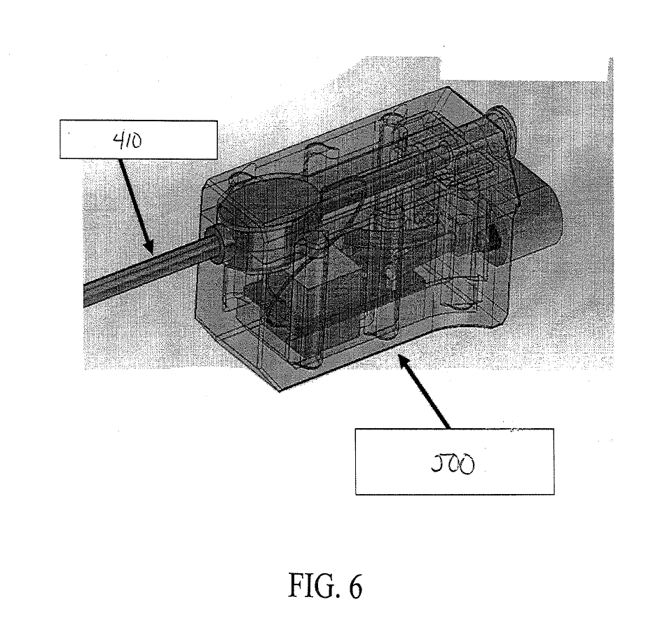

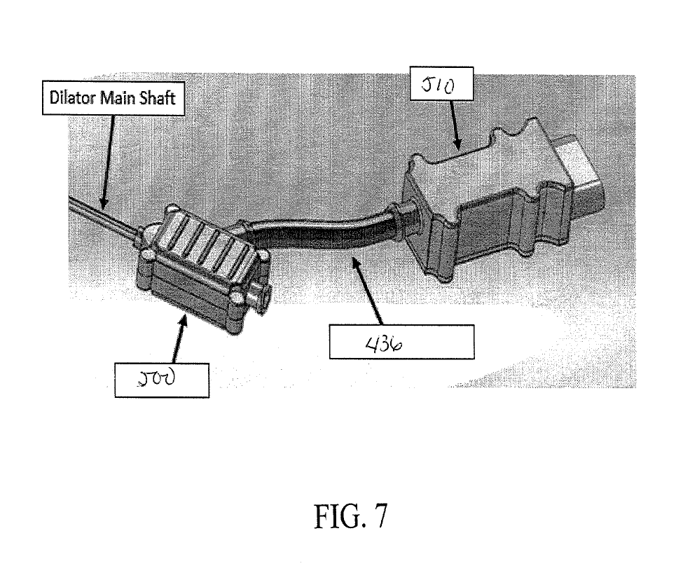

Referring now to FIG. 6 the dilator hub 500 of the dilator 400 in accordance with the invention is depicted. At the proximal end of the tubular main body 410, the dilator hub 500 is bonded or over-molded over the outer polymer body 413. In one aspect of the hub 500 and as best seen in FIG. 6, the electronics (handle card, connectors, etc.) are integrated or contained within the dilator hub 500 advantageously eliminating external cords that may interfere with the procedure during dilator and/or sheath manipulation. In a second aspect of the hub 500 as best seen in FIG. 7, the transmission lines 436 exit the dilator hub 500 and connect to a dongle 510 that contains the electronics.

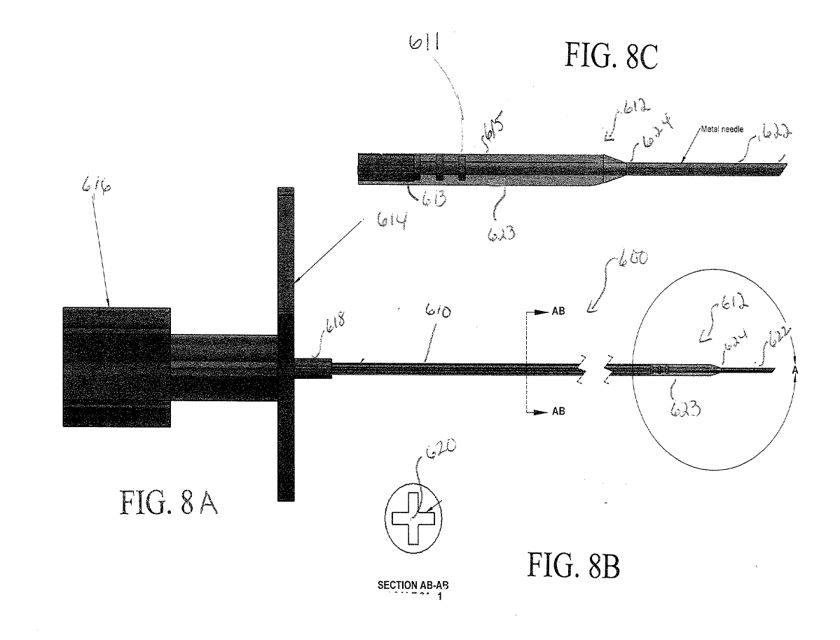

Referring now to FIG. 8, the transseptal puncture device 600 in accordance with the invention is depicted. The transseptal puncture device 600 in accordance with the invention includes shaft 610 operably coupled to needle portion 612, push plate 614 and housing 616. Shaft 610 is constructed from a material with a low electrical conductivity selected from polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and any combinations of the foregoing, and composites such as glass fiber and epoxy resin. As best seen in FIG. 8B, and as herein below described, shaft 610 may be extruded to include a lumen 620 that may comprise one of a number of particular geometric configurations (e.g. I-beam, X-beam, W-beam, etc.). Lumen 620 is extruded through the center axis of the shaft section 610 and the particular geometric configuration selected serves to increase the overall strength of the shaft 610.

Puncture plate 614 is operably coupled to shaft 610 by connector 618. Those of skill in the art will appreciate that shaft 610 may also be integrally coupled to puncture plate 614 or removably coupled via connector 618. Shaft 610 and connector 618 may be constructed from the same or different materials. In another aspect of the invention connector 618 and shaft 610 may be integrally formed. In another aspect of the invention, connector 618 provides added strength to the interface where the shaft joins to the puncture plate 614 and functions as a strain relief.

Housing 616 is operably coupled to puncture plate 614 and houses an insertion depth sensor (not shown) that provides information to the physician about how deep the transseptal needle has been inserted into the dilator. Housing 616 may also house transformers and other electronics that provide tactile feedback to the physician about needle depth. For example, the needle handle may be structured to vibrate as the needle is about to exit the dilator tip.

The transseptal puncture device 600 may be constructed from a single material or a combination of materials. Suitable materials include rigid non-conductive materials such as carbon fiber composites, glass fiber reinforced epoxy resin, polyether ether ketones (PEEK), polyetherimides (Ultem), polycarbonates and the like. In combination, the shaft 610 and needle portion 612 may prevent MR induced heating at the needle, for example if the bulk of the transseptal needle is non-conductive and the length of the conductive section is sufficiently small.

In one aspect of the invention, as best seen in FIG. 8C, the shaft 610 may consist of an inner layer 613 of rigid material such as PEEK, Ultem, high density polyethelenes, higher durometer Pebax, polycarbonates, Hytrel and an over-molded outer layer 615 of a softer and/or lubricious material such as low density polyethelenes, polyurethanes, silicone and lower durometer Pebax. Alternatively, the outer layer 615 may be coated with a lubricious coating known to those of skill in the art. The rigid material of the inner layer 613 thereby provides sufficient translation and rotational force, while the softer outer layer 615 provides flexibility and smooth insertion through tissue and organs and the like. In alternative embodiments, both the inner and outer layers may be made of rigid material to maximize total rigidity. The outer layer 615 may also maintain the shaft 610 intact, for example in situations where stress and/or other mechanical forces may cause the inner material to crack or break.

In another aspect of the invention, and as best seen in FIG. 8B, the shaft lumen 620 (in single layer constructs or the inner layer 613 in a dual-layer construct) may be formed in a specific shape to provide strength. For example, an X shape may be used or an I-beam shape or a W-shape in cross-section. The different geometric shapes may be in addition to using a rigid inner material or in lieu of using a rigid inner material. As shown in FIG. 8B one desired shape, shown as a cross or X-shape, is extruded through the center axis to increase the strength of the shaft section. As those of skill in the art will appreciate, different geometries may create different properties that are desired under varying circumstances.

Referring now to FIG. 8C a detailed view of one aspect of the needle portion 612 of the transseptal needle 600 in accordance with the invention will now be described. Needle portion 612 includes a puncture tip 622 at a distal end thereof. Shaft 610 includes a distal portion 623 that is over-molded or bonded onto the proximal section 624 of needle portion 612. As can be seen in FIG. 8C shaft 610 may also include an inner polymer layer and an outer polymer layer. Concentric anchor rings 611 comprise part of the proximal geometry of the metal needle portion 622 to help improve the tensile strength of the over mold bond.

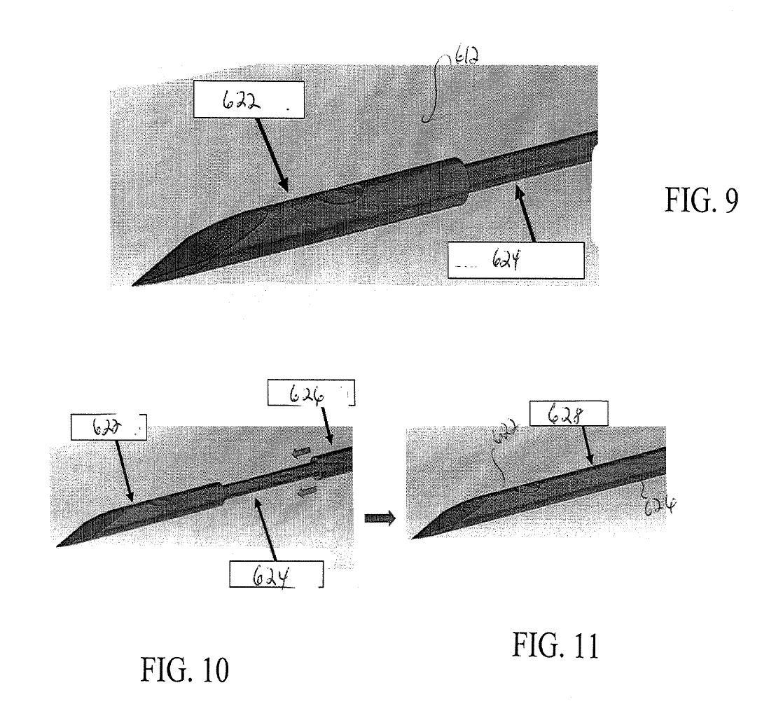

Referring now to FIGS. 9 through 11 various aspects of the needle portion 612 will now be described. As best seen in FIG. 9, needle 622 is shown as being over-molded or bonded onto first cannula 624. Bonding may include chemical or mechanical bonding techniques known to those of skill in the art. First cannula 624 includes an outer diameter that is smaller than the inner diameter of puncture tip/needle 622. Cannula 624 is constructed from materials selected from glass fiber reinforced epoxy composite, polyimide coated silica, including polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and combinations of the foregoing.

Puncture tip/needle 622 may be constructed from materials selected from polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and combinations of the foregoing. The puncture needle 622 may also be of conventional metal construction, such as stainless steel, titanium, nonmagnetic, nickel-cobalt-chromium-molybdenum alloys (such as MP35N), Nitinol, and other similar biocompatible metals, with an overall length of 4 inches or less, or constructed of a rigid non-conducting material as describe above. In another aspect, the needle 622 may be constructed using a metal having a polymer shaft over molded onto the needle as best seen in FIGS. 2 and 8C. In alternative aspects, the needle 622 may include ribs, barbs, or other mechanical features that work to secure the needle within the over molded polymer shaft.

After puncture tip 622 is over-molded or bonded to first cannula 624, a second cannula 626 is slidably positioned over first cannula 624. The inner diameter of second cannula 626 is greater than the outer diameter of first cannula 624 while the outer diameter of second cannula 626 is substantially equal to the outer diameter of puncture tip/needle 622 to ensure a tight bond between them and to ensure that a continuous outer surface is formed. Second cannula 626 is slidably received by first cannula 624 until the distal portion of the second cannula 626 abuts the proximal surface of the puncture tip 622 at point 628 best seen in FIG. 11. The outer cannula may be constructed from a material selected from polyimides, long-chain aliphatic polyamides such as GRILAMID (EMS-GRIVORY), thermoplastic elastomers including polyether block amides such as PEBAX (Arkema) and polyester elastomers such as HYTREL (Dupont), and combinations of the foregoing. The outer cannula 626 and puncture tip 622 may also be coated with a lubricious coating. The outer cannula 626 may be bonded or reflowed to the proximal surface of the puncture tip 622.

In other aspects of the transseptal needle, the cannula may be solid or hollow. The cannula may be made of MRI compatible materials such as PEEK, Ultem, Polycarbonate, or Glass Fiber reinforced Epoxy. If the cannula is hollow, it may be constructed to have a lumen that connects the distal puncture tip to the proximal handle. The distal puncture tip geometry could be created by a grinding process to create many different geometries. Alternatively, the distal puncture tip could be created by bonding or over molding a separate sharp component to the cannula. This bonded component could be solid or have a through hole. If the bonded component is solid, there could be flush holes proximal of the solid tip.

The transseptal needle tip in accordance with the invention could be passively tracked. The transseptal needle tip may be tracked by having a small metal component that interferes with the dilators tracking component and thereby indicates that the transseptal tip is passing through the dilator tracking region. The transseptal needle tip may also incorporate an active tracking region in a similar fashion to the dilator. (Tip coils, Flat Flex Circuit, etc . . . )

The transseptal needle may be tracked with a depth sensor that indicates the linear position of the needle in relation to the dilator. This information indicates the translational position of the needle tip in relation to the dilator, effectively tracking the needle. The depth sensor may be located proximally in the needle hub or dilator hub. Conversely, the depth sensor may be located distally in the needle tip or dilator tip.

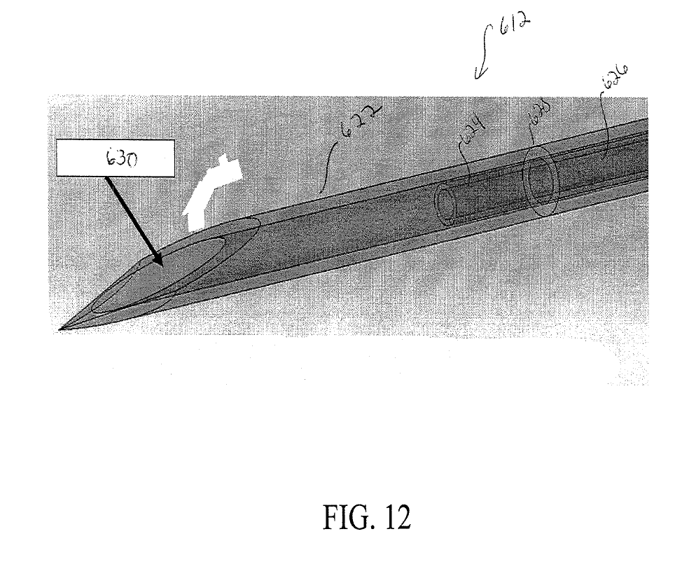

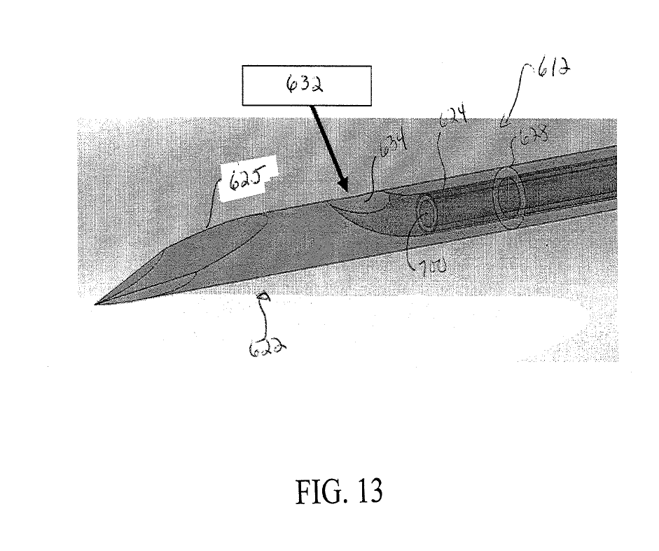

Referring now to FIGS. 12 and 13 various aspects of the puncture tip 622 in accordance with the invention will be disclosed. In one aspect, as best seen in FIG. 12, the puncture tip 622 includes ‘through hole’ 630 that is coaxial with first cannula 624 and co-extensive with the lumen 620 of the transseptal needle shaft 610 and provides an exit point at the tip 622 for guide wires and/or contrast media that may be inserted through the lumen of the transseptal needle shaft 610 to allow the physician to confirm left atrial cannulation after septum puncture.

In another aspect of the puncture tip 622, as best seen in FIG. 13, the distal tip portion 625 of the needle 622 comprises a solid construct. Proximal of the solid tip portion 625, a flush hole/channel 632 is continuous with the lumen 700 of first cannula 624 (and co-extensive with the lumen 620 of shaft 610) and includes an exit hole 634 positioned to the side of the puncture tip 622. Flush hole with its channel 632 serve as an exit conduit for contrast media or a guidewire. The contrast media or guidewire allow the physician to confirm left atrial cannulation after septum puncture.

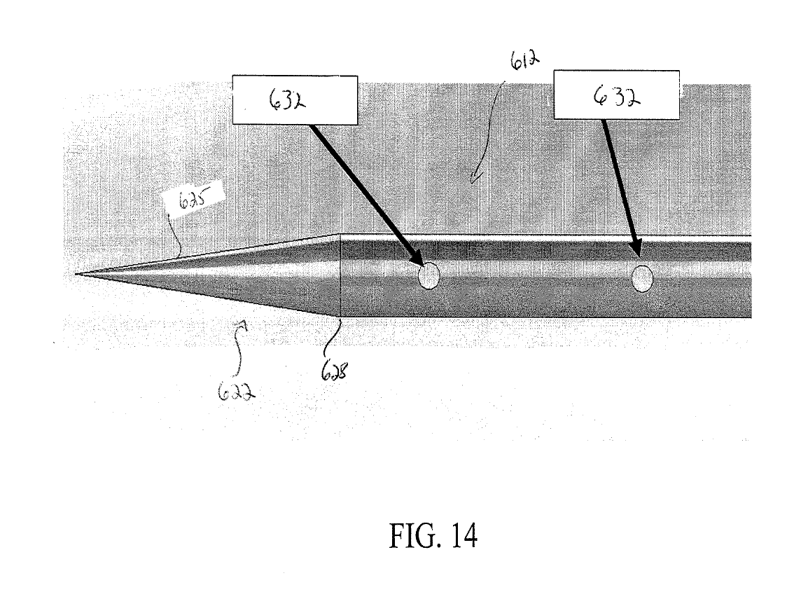

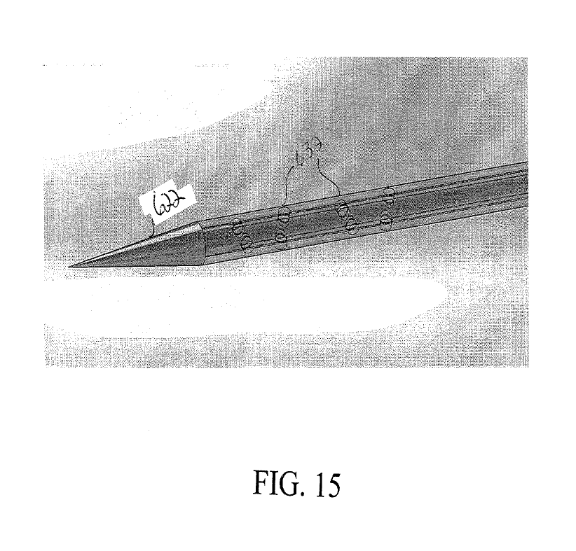

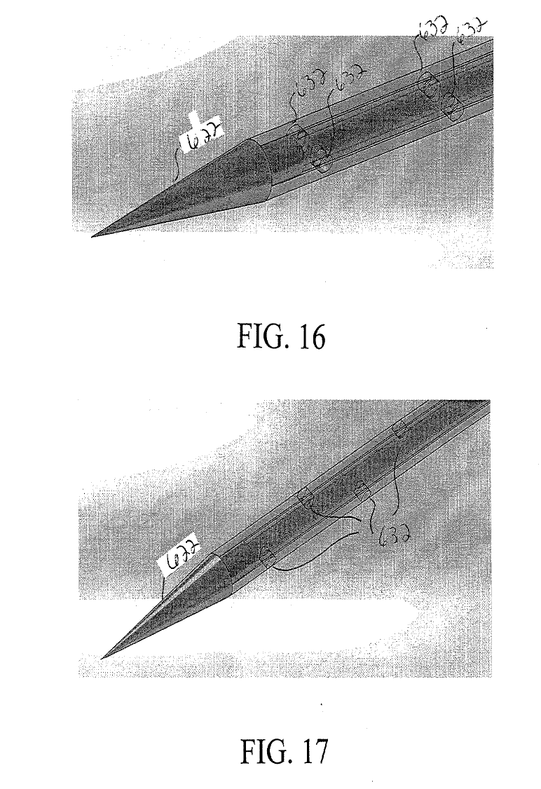

In yet other aspects of the puncture tip 622 in accordance with the invention, and as best seen in FIGS. 14 through 17, the distal portion 625 of puncture tip 622 is completely solid and has a conical shape. Those of skill in the art will appreciate, however, that distal tip 625 can be of any shape such as fluted, triangular, trocar-shaped, chiseled without departing from the invention so long as the tip is capable of piercing through tissue. Proximal of the puncture tip 622 are one or more flush holes that penetrate both the first and second cannulas 624, 626 to access their lumens such that contrast media can be injected into the needle and exit at these locations. The flush hole locations may be drilled in a variety of patterns and configurations including 90 degree staggered (FIG. 15), 180 degree opposed (FIG. 16), 180 degree staggered (FIG. 17) and like configurations. Those of skill in the art will also appreciate that any number of flush holes may be included on the shaft and the number is not limited to the number shown.

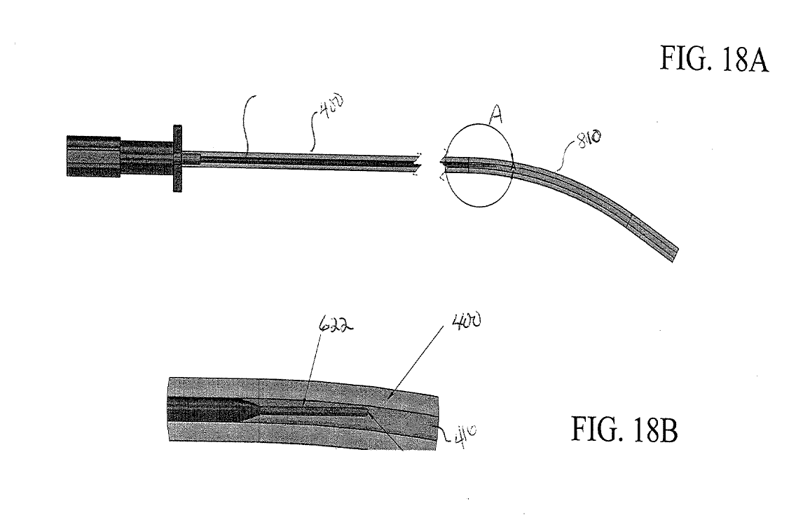

Referring now to FIG. 18 the transseptal puncture device in combination with the dilator is depicted. The transseptal puncture device 600 in accordance with the invention may be constructed such that it can be introduced through the lumen 416 of the dilator 400 hereinbefore described. The puncture device may include an optional lumen through which a stylet can be passed. In another aspect of the transseptal puncture device the puncture device may not include a lumen. The needle may be sufficiently short to prevent it from contacting the inner walls of the dilator lumen, thereby eliminating the need for a stylet. If a stylet is used, the stylet may be constructed from a polymer or similar material.

As best seen in FIGS. 18A and 18B, in operation the transseptal needle device 600 is placed inside the lumen 416 of dilator 400. The distal portion 810 of the dilator 400 is bendable. A bendable dilator distal section allows the physician to create a more patient specific curve for better directing the tip of the sheath/dilator/needle assembly to the puncture target.

The transseptal puncture device may use one or more methods of MR tracking. In a first aspect, the actively tracked sheath and/or dilator described above may be utilized. In this first aspect, the transseptal puncture device is used in conjunction with the actively tracked sheath and/or dilator with lumen. A tracking coil is integrated into the dilator and the transseptal needle is inserted through a lumen in the dilator and tracked in the method described above.

In another aspect, MR tracking may consist of visualization of the needle using passive markers. In yet another aspect, a coupling between an active coil in the dilator and a metallic needle on the device may be provided. In another aspect of tracking, an electronic sensor may be integrated into proximal region of the needle shaft or needle handle to determine the needle penetration depth and the position of the needle tip relative to the location of the tracking coils in the dilator. In another aspect of tracking, an electronic sensor may be integrated in the distal region of the needle tip or dilator to determine the relative position of the needle tip in relation to the dilator tip. The transseptal puncture device in accordance with the invention may be used to puncture the septum of the heart. To determine when the septum has been crossed, and to determine the pressure applied to the septum, a pressure sensor, such a fiber optic Bragg grating, may also be placed in the distal needle portion 622 of the device 600.

Although the present invention has been described with reference to preferred embodiments, those of ordinary skill in the art will recognize that changes may be made in form and detail without departing from the spirit and scope of the invention.

1. An actively tracked medical device comprising:
a dilator having an inner tubular main body having a distal end and a proximal end, said tubular main body including at least first and second receiving channels positioned in a spaced apart relationship on an outer surface of said tubular main body;
a region at the distal end of said tubular main body for supporting one or more tracking coils;
an atrumatic tip portion operably coupled and positioned distal to said main body;
a lumen extending through said tubular main body, said tip support and said atraumatic tip portion; and
an outer polymer body having first and second ends, said outer polymer body operably covering said inner tubular main body and said tracking coils, said first end terminating adjacent a proximal end of said atraumatic tip portion and said second end terminating adjacent said hub.

2. The actively tracked medical device of claim 1 wherein one or both of said first and second channel lumens receive a transmission line, a co-axial cable, transmission line transformers and one or more matching network cards.

3. The actively tracked medical device of claim 1 wherein said lumen is adapted to receive a needle, stylet, a guidewire, fluid or contrast media.

4. The actively tracked medical device of claim 1 wherein said tubular main body comprises a multi-lumen extrusion construct.

5. The actively tracked medical device of claim 1 wherein said region at the distal end of the tubular main body includes grooves to receive said tracking coils.

6. The actively tracked medical device of claim 2 further comprising a hub for housing electronics, said hub operably coupled to one or more of said transmission lines selected from co-axial cables, transformers and a matching network card.

7. The actively tracked medical device of claim 1 wherein said dilator is disposed within an MR compatible steerable sheath.

8. The actively tracked medical device of claim 7 wherein said sheath includes a metal foil ring at a distal end thereof for reducing the tracking signal of an inserted trackable device for the purpose of identifying and/or tracking the tip of the sheath in an MR environment.

9. The actively tracked medical device of claim 1 further comprising a transseptal needle device, said transseptal needle including a cannula portion having proximal and distal ends, a push plate operably coupled to said proximal end and a needle portion operably coupled to said distal end.

10. The actively tracked medical device of claim 9 wherein said shaft portion includes a lumen through a center axis thereof, said lumen having an I-beam, X-beam or W-beam configuration.

11. The actively tracked medical device of claim 9 further comprising a housing operably coupled to said push plate, said housing containing a depth sensor therewithin.

12. The actively tracked medical device of claim 9 wherein said shaft comprises an inner layer of rigid material and an outer layer of a softer material.

13. The actively tracked medical device of claim 9 wherein said needle portion comprises a needle puncture tip.

14. The actively tracked medical device of claim 13 wherein said needle puncture tip is solid or includes lumen therethrough.

15. The actively tracked medical device of claim 14 wherein said needle puncture tip is solid and includes on or more flush channels comprising an exit conduit for contrast media or a guidewire.

16. The actively tracked medical device of claim 13 wherein said needle puncture tip has a configuration selected from conical, fluted, triangular, trocar, or chisel shaped.

17. The actively tracked medical device of claim 15 wherein said one or more flush channels are drilled on said needle puncture tip in a configuration selected from 90 degree staggered, 180 degree opposed or 180 degree staggered.

18. The actively tracked medical device of claim 9 wherein said distal portion of said shaft is over-molded or bonded onto said needle portion.

19. The actively tracked medical device of claim 9 wherein said needle portion further comprises a needle puncture tip, a first cannula having an outer diameter that is smaller than an inner diameter of said needle puncture tip, said first cannula operably coupled to said needle puncture tip.

20. The actively tracked medical device of claim 19 further comprising a second cannula in co-axial relationship with said first cannula, said second cannula operably coupled to said needle puncture tip.

21. The actively tracked medical device of claim 9 wherein a portion of said tubular main body of said dilator is deflectable.

22. The actively tracked, medical device of claim 21 wherein said transseptal needle device is configured to be received within said dilator and said needle portion is shorter than said deflectable main body portion.

23. The actively tracked medical device of claim 9 further comprising an actively tracked needle.

24. An actively tracked medical device comprising:
a dilator in combination with a transseptal needle wherein said dilator comprises an inner tubular main body having a distal end and a proximal end, said tubular main body including at least first and second receiving channels positioned in a spaced apart relationship on an outer surface of said tubular main body;
a region at the distal end of said tubular main body intended to support;
one or more tracking coils;
an atrumatic tip portion operably coupled and positioned distal to said distal end of the main body;
a lumen extending through said tubular main body, said tip support and said atraumatic tip portion;
an outer polymer body having first and second ends, said outer polymer body operably covering said inner tubular main body and said tracking coils, said first end terminating adjacent a proximal end of said atraumatic tip portion and said second end terminating adjacent said hub; and
wherein said transseptal needle comprises a shaft portion having proximal and distal ends, a push plate (or handle) operably coupled to said proximal end and a needle portion operably coupled to said distal end.

25. The actively tracked medical device of claim 23 wherein said needle includes integrated tracking coils or wherein said needle acts as a receiving antenna for active tracking.

26. An actively tracked medical device comprising:
a dilator having a distal tip, a tracking region thereon, a proximal hub, and a main shaft that operably couples the tip of the dilator to the hub;
a means for communicating with the tracking region.

27. The actively tracked medical device of claim 26 wherein said distal tip is atraumatic.

28. The actively tracked medical device of claim 27 where said distal tip includes a gradual taper angle or is constructed from a soft material or both.

29. The actively tracked medical device of claim 26 wherein the tracking region includes a tip support that supports wound tracking coils or PCB tracking coils or matching network cards or combinations of the foregoing.

30. The actively tracked medical device of claim 26 wherein the tracking region comprises a flat flex circuit that integrates the tracking coils and matching network cards.

31. The actively tracked medical device of claim 26 wherein the means for communicating with the tracking region comprises transmission lines that operably couple the tracking region to said hub.

32. A transseptal needle comprising a cannula portion having proximal and distal ends, a push plate operably coupled to said proximal end; a needle portion operably coupled to said distal end.

33. The transseptal needle of claim 32 wherein said transseptal needle includes tracking means thereon.

34. The transseptal needle of claim 32 wherein said needle is passively tracked.
